(19) United States
(12) Patent Application Publication
(10) Pub. No.: US 2005/0137517 A1
(43) Pub. Date: Jun. 23, 2005

(54) Processing systems and methods for providing leukocyte-reduced blood components conditioned for pathogen inactivation

(75) Inventors: Bryan J. Blickhan, Zion, IL (US); Mary Stewart-Wesson, Mundelein, IL (US); Daniel Bischof, Bull Valley, IL (US); Mohsen Arghavani, Mount Prospect, IL (US); Lawrence J. Servi JR., Hawthorn Woods, IL (US); Daniel Lynn, Spring Grove, IL (US); Tat Mui, Niles, IL (US); Michael C. Mayo, Mundelein, IL (US)

Correspondence Address: BAXTER HEALTHCARE CORPORATION;Bradford R.L. Price, Fenwal Division RLP-30, Route 120 and Wilson Road, Round Lake, IL, 60073, US

(73) Assignee: Baxter International Inc.

(21) Appl. No.: 10/741,583

(22) Filed: Dec. 19, 2003

(51) Int. Cl. A61M 37/00

(52) U.S. Cl. 604/6.03

(57) ABSTRACT

Systems and methods process blood and blood components for subsequent pathogen inactivation processes prior to long term storage and/or transfusion.

### RELATED APPLICATIONS

This application claims the benefit of the priority date of copending U.S. patent application Ser. No. 10/008,361, filed Dec. 5, 2001 and entitled “Manual Processing Systems and Methods for Providing Blood Components Conditioned for Pathogen Inactivation.”

### FIELD OF THE INVENTION

The invention generally relates to the processing of whole blood and its components for storage, fractionation, and transfusion.

### BACKGROUND OF THE INVENTION

The clinically proven components of whole blood include, e.g., red blood cells, which can be used to treat chronic anemia; plasma, which can be used as a blood volume expander or which can be fractionated to obtain Clotting Factor VIII-rich cryoprecipitate for treatment of hemophilia; and concentrations of platelets, used to control thrombocytopenic bleeding.

Along with the growing demand for these blood components, there is also a growing expectation for purity of the blood product. Before storing blood components such as red blood cells or platelets for later transfusion, it is believed to be desirable to minimize the presence of impurities or other materials that may cause undesired side effects in the recipient.

For example, it is generally considered desirable to remove leukocytes from such blood components before storage, or at least before transfusion. It is also believed beneficial that potential blood-born pathogens, e.g., viruses and bacteria, be inactivated from blood components prior to transfusion, e.g., through the use of photoactive and non-photoactive chemical reactions.

### SUMMARY OF THE INVENTION

The invention provides systems and methods for processing concentrated red blood cells and the like. The systems and methods condition the concentrated red blood cells for subsequent pathogen inactivation processes prior to long term storage and/or transfusion.

The systems and methods condition a collected concentration of red blood cells (called “packed red blood cells or pRBC's) for a pathogen inactivation function, which removes and/or inactivates suspected pathogens prior to long term storage. The systems and methods include a leukocyte reducing function, which reduces the residual population of leukocytes in the pRBC's prior to pathogen inactivation. In one embodiment, the leukocyte removing function is accomplished by filtration. The systems and methods also add a synthetic conditioning solution to the pRBC's. The conditioning solution is selected to specially condition the pRBC's for pathogen inactivation, in terms of, e.g., desired viscosity and/or desired physiologic conditions, such as pH. The systems and methods include a dilution function, during which at least one component of a conditioning solution is mixed with the pRBC's prior to the leukocyte reducing function. The component lowers the viscosity of the pRBC's and can lead to higher flow rates during filtration.

It has been discovered that, when the component of the conditioning solution, as conventionally formulated, is added to the pRBC's during the dilution function, a degradation of filtration efficiencies results when certain filtration media are used. When such filtration media are used, there is an observed increase in the residual leukocyte population and/or a decrease in the recovery following filtration of the pRBC's after filtration in the presence of this conventionally formulated conditioning solution component, when compared to the residual leukocyte population and/or pRBC recovery in red blood cells filtered by the same filters when mixed with other typical additive solutions commercially available today.

Based upon this surprising discovery, it is believed desirable to differentiate between filtration media having performance characteristics that are adversely affected by exposure to the conventionally formulated component of the conditioning solution (which will be called Category A Filtration Media) from filtration media that are not adversely affected (which will be called Category B Filtration Media).

Accordingly, one aspect of the invention provides systems and methods that include an identification function, by which filtration media having performance characteristics that are adversely affected by exposure to the conventionally formulated conditioning solution (Category A Filtration Media) are identified and differentiated from filtration media that are not so adversely affected (Category B Filtration Media). Once identified, Category B Filtration Media can be selected to perform the leukocyte reducing function, if desired.

However, Category A Filtration Media can still be selected to perform the leukocyte reducing function, if desired. According to this aspect of the invention, the systems and methods counteract the expected degradation of leukocyte removal efficiency in various ways.

For example, the systems and methods can provide a higher osmolarity for the component of the conditioning solution added before and/or during filtration. A higher osmolarity means exposure of the pRBC's to less hypotonic conditions prior to and/or during filtration. Reducing the hypotenicity of the conditioning solution component can be accomplished in various ways, e.g. by the addition of dextrose, and/or the addition of sodium chloride, and/or by the retention of greater volumes of anticoagulated plasma with the pRBC's.

As another example, the systems and methods can raise the extracellular pH of the pRBC's prior to and/or during the leukocyte reducing function. The pH can be elevated in various ways, e.g., by revising the content of phosphate in the conditioning solution, or chilling the pRBC's prior to filtration.

As another example, the systems and methods can meter the introduction of the hypotonic component of the conditioning solution during the leukocyte reducing function. In this arrangement, exposure of the pRBC's to the component is reduced prior to and/or during the leukocyte reducing function.

Another aspect of the invention provides systems and methods that include a pump to convey pRBC's through the filter during the leukocyte reducing function. The pump reduces the time of exposure during filtration to conditions that may possibly degrade filtration efficiencies. Improved leukocyte-reduction may also result when a pump is used, due to the effect of pump-induced shear forces on the blood, which can stimulate platelet and/or leukocyte adhesion to the filtration media.

### DESCRIPTION OF THE DRAWINGS

FIG. 1 is a diagrammatic view of a system and related method for treating a collected concentration of red blood cells to remove and/or inactivate suspected pathogens prior to long term storage and/or transfusion to a patient.

FIG. 2 is a view of a representative system for carrying out a leukocyte reducing function and the conditioning function of the method shown in FIG. 1 using a hypotonic Esol-A component.

FIG. 3 is a view of a representative system for carrying out a dilution function of the method shown in FIG. 1, which coordinates a conditioning function using a hypotonic Esol-A component with a leukocyte reducing function.

FIG. 4 is a view of a representative system for carrying out a dilution function of the method shown in FIG. 1, which coordinates a conditioning function using a modified, less hypotonic (e.g., having a higher ostmotic strength) Esol-A component with a leukocyte reducing function.

FIG. 5 is a view of a representative system for carrying out a dilution function of the method shown in FIG. 1, which coordinates a conditioning function with a leukocyte reducing function, and which includes the use of an additive solution that is ultimately replaced by a hypotonic Esol-A component.

FIG. 6 is a view of a representative system for carrying out a conditioning function of the method shown in FIG. 1 on a pre-processed, leukocyte-reduced pRBC unit.

FIG. 7 is a view of a representative system for carrying out a dilution function of the method shown in FIG. 1, which coordinates a conditioning function using a hypotonic Esol-A component with a leukocyte reducing function, and which includes pump-assisted flow.

FIG. 8 is a view of a representative system for carrying out a dilution function of the method shown in FIG. 1, which coordinates a conditioning function using a hypotonic Esol-A component with a leukocyte reducing function, and which includes metered flow of the hypotonic Esol-A component.

The invention is not limited to the details of the construction and the arrangements of parts set forth in the following description or shown in the drawings. The invention can be practiced in other embodiments and in various other ways. The terminology and phrases are used for description and should not be regarded as limiting.

### DESCRIPTION OF THE PREFERRED EMBODIMENTS

I. System Overview

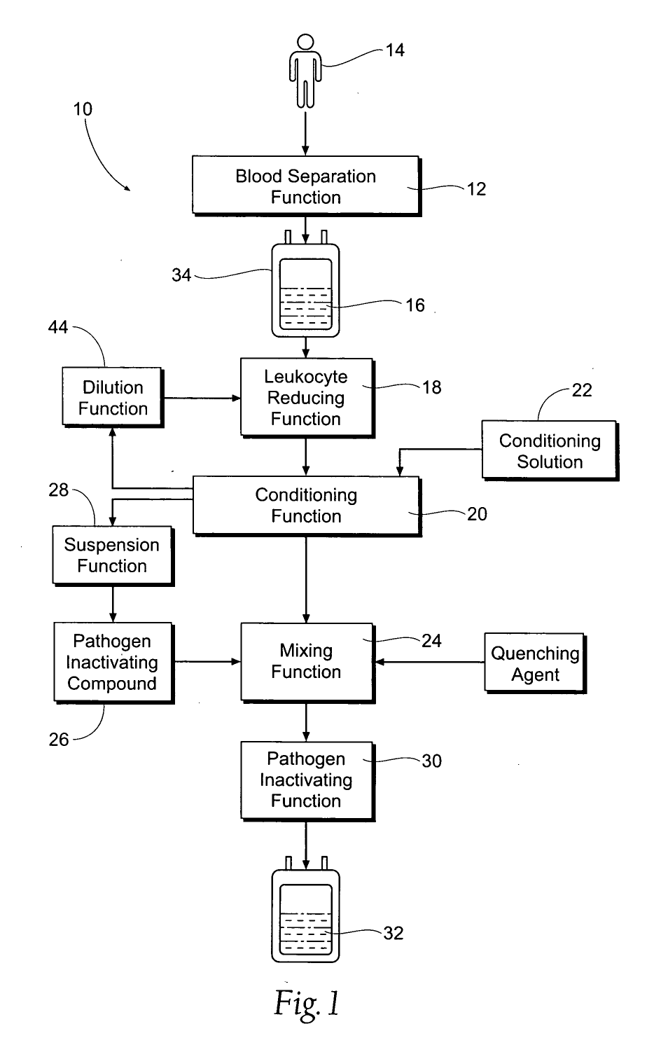

FIG. 1 shows an overview of a system and related method 10 for treating a collected concentration of red blood cells B which can also be called Apacked@ red blood cells, or pRBC—to remove and/or inactivate suspected pathogens prior to long term storage and/or transfusion to a patient. Blood-borne pathogens can include a multitude of bacterial and/or viral agents such as, for example, hepatitus B virus or human immunodeficiency virus. It is not desirable to expose a patient in need of blood transfusion therapy to such pathogens, and it is the purpose of the system and related method 10 to minimize the possibility of this event.

The system and related method 10 include a blood separating function 12. The function 12 processes whole blood drawn from a donor 14 to generate a pRBC unit 16. The function 12 can comprise the use of conventional closed, sterile, manually manipulated blood collection systems, such as the Blood Pack® Units manufactured and sold by Baxter Healthcare Corporation, Deerfield, Ill. The use of manual systems will typically yield a pRBC unit 16 containing a unit volume of pRBC's of about 150 to 300 ml. Alternatively, the function 12 can comprise the use of automated blood collection systems, such as the Amicus™ Blood Collection System, or the Alyx™ Blood Collection System, of the CS-30007 Blood Collection System, all of which are manufactured and sold by Baxter Healthcare Corporation, Deerfield, Ill. The use the Alyx™ Blood Collection System can yield a unit volume of pRBC's of upwards to about 500 ml, although the exact yield can vary. Further details of the system and related method 10 in these different blood collection environments will be described later.

The system and related method 10 also include a leukocyte reducing function 18. Reducing the residual population of leukocytes in a blood product collected for transfusion is recognized to be beneficial and is today recommended by most governmental agencies overseeing blood banking activities. For example, in the United States, for pRBC's to be considered “leukoreduced,” the regulatory requirement is the reduction of the residual population of leukocytes in a given pRBC unit to less than 5×106 prior to transfusion. The aggressive reduction of leukocytes also serves the beneficial purpose of removing the total pathogen load of the blood component by first removing pathogens that may be entrained within leukocytes prior to pathogen inactivation.

In the illustrated embodiment (as will be explained later), the pRBC unit 16 is desirably passed through a filter to separate leukocytes from the red blood cells, e.g., by exclusion using a membrane or by depth filtration through a fibrous filter media. It should be appreciated, however, that leukocyte separation can occur by various centrifugal and non-centrifugal techniques, and not merely “filtration” in the technical sense. Separation can occur by absorption, columns, chemical, electrical, and electromagnetic means. “Filtration” is broadly used in this specification and encompasses all of these separation techniques as well.

The system and related method also include a conditioning function 20. It is desirable that the pRBC unit 16 be in a condition that facilitates a subsequent pathogen inactivation process, as well as long-term storage following pathogen inactivation. To this end, the conditioning function 20 adds a selected conditioning solution 22 to the pRBC unit 16. The conditioning solution 22 is selected to specially condition the pRBC unit 16 for pathogen inactivation in terms of, e.g., desired viscosity and/or desired physiologic conditions, such as pH, which are conducive to effective pathogen inactivation. The conditioning solution 22 also desirably conditions the pRBC unit 16 for long-term storage after pathogen inactivation, by providing the proper mix of nutrients and buffers to sustain blood cell metabolism during storage.

By way of example, a conditioning solution 22 of the type known as Erythro-sol™ (also known as E-Sol™) (sold by Baxter Healthcare Corporation), can be mixed with the pRBC unit 16 to condition it for pathogen inactivation, particularly when the selected pathogen inactivation agent includes the frangible compounds disclosed in Cook et al. U.S. Pat. No. 6,093,725. As conventionally formulated, E-Sol™ Solution comprises sodium citrate (25 mM); dibasic sodium phosphate (16.0 mM); monobasic sodium phosphate (4.4 mM); adenine (1.5 mM); mannitol (39.9 mM); and dextrose (45.4 mM). For practical reasons related to heat sterilization (more particularly, because dextrose will degrade under heat sterilization conditions if maintained at a pH above 7.0, which is the pH condition of the overall Esol™ Solution, which typically ranges from 7.0 to 7.5 and is preferably between 7.3 to 7.5), the conventional E-Sol™ Solution is typically added to red blood cells as two separate components—an Esol-A component and a dextrose solution (called an Esol-B component). As conventionally formulated, the Esol A component comprises 94 ml of sodium citrate (26.6 mM); dibasic sodium phosphate (17.0 mM); monobasic sodium phosphate (4.7 mM); adenine (1.6 mM); and mannitol (42.5 mM). The above compositions can be made by modifying the stated concentrations by +/−15%. The pH of the Esol A component generally matches the pH of the overall E-sol™ Solution. However, being free of dextrose, the Esol-A component can undergo heat sterilization. The Esol-B component comprises 20 ml of 8% dextrose. Being acidic, the Esol-B component can undergo heat sterilization separate from the components of the Esol-A component.

The system and related method 10 also include a pathogen inactivating compound mixing function 24. The mixing function 24 receives a pRBC unit 16 after it has undergone the leukocyte reducing function 18. When a two-part conditioning solution is used, the mixing function 24 can also be preceded by at least a portion of the conditioning function 20, which can occur before or as part of the leukocyte reducing function 18, as will be described in greater detail later. The mixing function 24 mixes the pRBC unit 16 with desired volume of a selected pathogen inactivating compound 26. As will be described later, when a two-part conditioning solution is used, at least a part of the conditioning solution 22 can be used as a suspension agent for the pathogen inactivating compound, as will be described later. The mixing function 24 ultimately generates a pRBC treatment unit, which comprises the leukocyte reduced pRBC unit 16 mixed with the conditioning solution 22 and pathogen inactivating compound 26.

In the illustrated embodiment, in which the conditioning solution 22 comprises the Esol™ solution, the pathogen inactivation compound 26 desirably comprises a pH-sensitive frangible anchor-linker-effecter (Frale) compound. This compound performs a pathogen inactivating function by irreversibly preventing the replication of DNA of blood borne pathogens. A pathogen inactivation compound 26 of this type is β-alanine, —N-(acridin-9-yl), 2-[bis(2-chloroethyl)amino]ethyl ester. This compound 26 and its use are described in more detail in U.S. Pat. Nos. 6,093,725 and 6,410,219 which are incorporated by reference herein.

A quenching agent, e.g., L-Glutathione, is desirably used in association with the pathogen inactivating compound 26 just described. L-Glutathione is a naturally occurring tripeptide that does not penetrate the red cell membrane and pathogen membrane and/or coat. The purpose of a quenching agent is to inhibit non-specific reaction of the pathogen inactivation compound 26 with nucleophiles other than DNA/RNA, as the mixing function 24 may provide excess pathogen inactivation compound 26 to assure complete reaction treatment of the red cells.

The system and related method 10 also includes a pathogen inactivating function 30. The pathogen inactivating function 30 carries out the steps required to complete the pathogen inactivation process of the pRBC treatment unit.

As above described, the pathogen inactivation compound 26 is pH-activated. At an acidic pH, the Frale compound 26 is inactive, and does not react excessively with the quenching agent. It can therefore be stored prior to use in an inactive state. However, when the compound 26 is added to the higher pH red cells during the mixing function 24, the pathogen inactivating compound 26 becomes activated to carry out the inactivation process, which carries forward into the inactivating function 30. Once activated by the proper pH conditions, the pathogen inactivation compound 26 becomes a highly reactive acridine based compound. During the subsequent inactivating function 30, the activated compound penetrates the red cell membrane, pathogen membrane and/or coat and, through a reactive intermediate, cross links the nucleic acids and pathogens. The cross links inactivate the pathogens by preventing replication of their genomes.

The pathogen inactivating function 30 desirably subjects the combined collected red blood cells, inactivation compound 26, and quenching agent (along with the conditioning solution 22) to further mixing, e.g., by passage through a static mixer. This assures that all the red blood cells have been treated by the inactivation agent. After mixing, the pathogen inactivating function 30 desirably also incubates the combined collected red cells, pathogen inactivation compound 26, and quenching agent (along with the conditioning solution 22) for a period of time sufficient to assure that the inactivation process has taken place. Presently, it is contemplated that incubation in an environmentally controlled area having an ambient temperature of between about 19-25° C., for about 1-24 hours and more preferably 12 hours, is sufficient, although more or less time may be required. After incubation, the pathogen inactivating function 30 desirably treats the combined red cell and inactivation compound 26 to remove any unused pathogen inactivation compound 26 and any reaction or degradation product of the compound 26. In a preferred embodiment, the combined solution is contacted with a sorption device, which may operate by adsorption, absorption or other sorption mechanisms to cleave to or otherwise remove any remaining inactivation agent and degradation or reaction by-products.

The pathogen inactivating function 30 ultimately generates a pathogen inactivated PRBC unit 32. The pathogen inactivated unit 32 is ready to undergo long term storage and/or transfusion to a patient.

II. Coordination of the Conditioning Function

Desirably synergistic effects can be achieved by the purposeful coordination of the conditioning function 20 with the mixing function 24 and/or the leukocyte reducing function 18.

A. Coordination of the Conditioning Function and the Mixing Function

As described above, the pathogen inactivation compound 26 may be provided in an inactive, ready-to-use liquid form or may alternatively be provided in a concentrated form which requires reconstitution or other processing before addition to the red cells. For example, the pathogen inactivation compound 26 as above described may be provided in the form of a crystalline powder, a granulated powder, tablet, capsule, lyophilized powder, concentrated liquid or frozen liquid. The compound 26 may be supplied in a wide variety of containers, such as bags, vials, rigid or flexible, syringe, or tubing or other appropriate container. In a preferred embodiment, about 10-100 mg, and more preferably about 50 mg of the compound 26 in dry powder form is contained in a vial or other suitable container. The quenching agent L-Glutathione may also be provided in various formulations and forms, including crystalline powder, liquid, low pH liquid, granulated powder, tablet, capsule, lyophilized powder or frozen liquid and may come in the same variety of containers as the pathogen inactivation agent. In a preferred embodiment as now contemplated, about 250-400 mg, and more preferably about 312 mg of L-Glutathione are provided in a vial or other suitable container.

The system and related method 10 shown in FIG. 1 include a suspension function 28. The suspension function 28 coordinates the conditioning function 20 with the mixing function 24.

The suspension function 28 introduces the acidic (low pH) Esol-B component into the pathogen inactivating compound 26 prior to the addition of the inactivating compound 26 (now suspended in the Esol-B component) to the pRBC unit 16 (now in a leukocyte-reduced state). The low-pH Esol-B component does not activate the pathogen inactivating compound 26. The suspension function 28 thereby allows the conditioning function 20 to augment the mixing function 30, by suspending the pathogen inactivation compound 26 (and the quenching agent) without activating the compound 26.

The suspension function 28 desirably carries out reconstitution of the pathogen inactivating compound 26 by repeated circulating the low pH Esol-B component into and out of the vial or container carrying the dry inactivation compound 26, until the compound 26 is suspended or dissolved in the Esol-B component. The inactivating compound 26 and Esol-B component are then repeatedly injected into and withdrawn from the vial of container holding the quenching agent, until the quenching agent is also resuspended in the Esol-B component. The Esol-B component with the inactivating compound 26 and quenching agent are now ready to be added to the pRBC's, which desirably have already been treated to remove leukocytes during the leukocyte reducing function 18. Alternatively, the quenching agent can be reconstituted first, followed by the reconstitution of the pathogen inactivating compound 26.

B. Coordination of the Conditioning Function and the Leukocyte Reducing Function

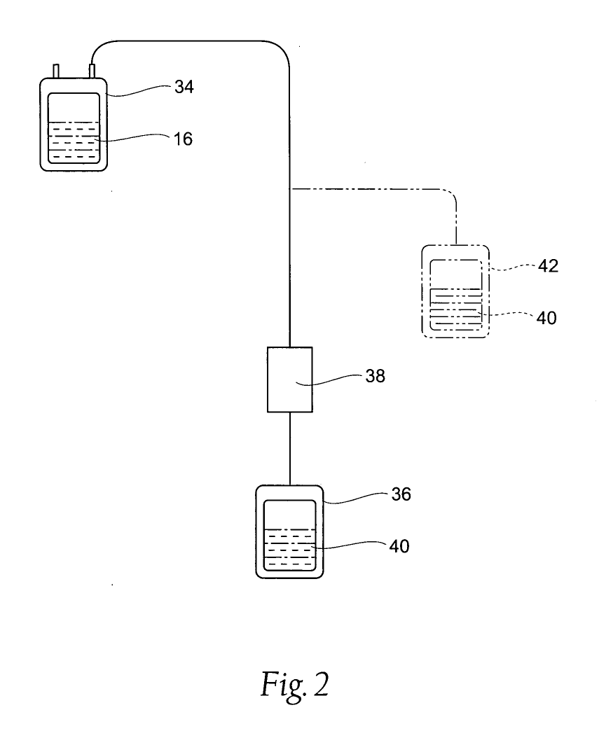

In a representative embodiment (see FIG. 2), the leukocyte reducing function 18 can be accomplished by passing the pRBC unit 16 from a collection container 34 (in the presence of an appropriate anticoagulant) to an integrally connected transfer container 36 through an in line leukocyte filter 38, e.g., by gravity flow. In this arrangement, the transfer container 34 holds the requisite volume of the Esol-A component 40, which mixes with the leukocyte-reduced pRBC's conveyed into the transfer container 36.

Alternatively (as shown in phantom lines in FIG. 2), the Esol-A component 40 can be transferred into the transfer container 36 from an auxiliary container 42 after conveyance of pRBC's into the container 36 through the filter 38 (in the presence of an appropriate anticoagulant). In this arrangement, the auxiliary container 42 is preferably coupled (by an integral connection or by use of a sterile docking technique) upstream of the filter 38 (as shown in phantom lines in FIG. 2), so that the Esol-A component 40 passes through the filter 38, flushing residual pRBC's from the filter 38, and mixing with the pRBC's in the transfer container 36.

In either situation, the Esol-B component is separately used as a suspension agent for the pathogen inactivating compound 26 and quenching agent, which is activated by its addition to the pRBC's and high pH Esol-A component 40 residing in the container 36 during the subsequent mixing function 24, as has been previously described.

It is desirable to lower the viscosity of the packed red blood cells before passage through the filter 38. The lower viscosity leads to higher flow rates, higher recovery of red blood cells, and a lower incidence of red blood cell damage or hemolysis. It is therefore desirable to add a viscosity-reducing solution to the red blood cells prior to filtration.

The system and related method 10 shown in FIG. 1 includes a dilution function 44. The dilution function 44 coordinates the conditioning function 20 with the leukocyte reducing function 18, by introducing all or at least a portion of the Esol-A component to the pRBC unit 16 prior to and/or during the leukocyte reducing function 18. The dilution function 44 thereby allows the conditioning function 20 to augment the leukocyte reducing function 18, by reducing the red blood cell viscosity during the leukocyte reducing function 18, while also providing at least some of the conditioning effect of the conditioning function 20.

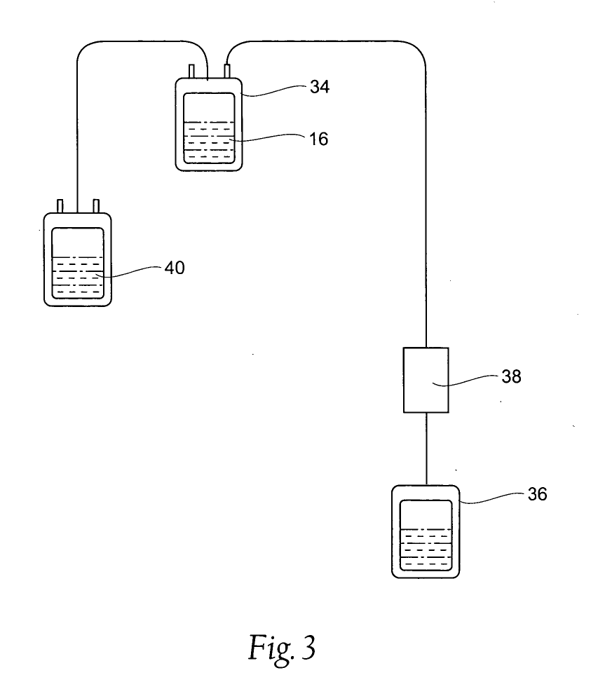

In a representative embodiment (see FIG. 3), to carry out the dilution function 44, an auxiliary container 42 holding the Esol-A component 40 can be coupled (either by an integral connection or by use of a sterile docking technique) to the collection container 34. As before described, the collection container 34 is itself connected to the transfer container 36 through the in-line leukocyte filter 38 (either by an integral connection or by use of a sterile docking technique). In this arrangement, the Esol-A component 40 is added to the pRBC unit 16 in the collection container 34 prior to filtration, thereby carrying the dilution function 44 in this embodiment. It is the lowered viscosity mixture of Esol-A component 40 and red blood cells that is passed through the filter 38 into the transfer container 36, e.g., by gravity flow.

In this arrangement, the Esol-B component is still used as the suspension agent for the pathogen inactivating compound 26 and quenching agent. The Esol-B component is added to the pRBC's and high pH Esol-A component 40 residing in the container 36 during the subsequent mixing function 24, as has been previously described.

Surprisingly, it has been discovered that, for certain types of leukocyte filtration media, the addition of the Esol-A component 40, as conventionally composed, to pRBC's prior to filtration, can result in a degradation of filtration efficiencies, such that there is an observed increase in the residual leukocyte population of the Esol-A pRBC's mixture after filtration, when compared to the residual leukocyte population in PRBC's filtered by the same filters when mixed with other typical additive solutions commercially available today.

For example, it has been observed that the residual leukocyte population in packed red blood cells is greater than expected and/or the RBC recovery is lower than expected when the pRBC's are filtered mixed with the Esol-A component through the fibrous depth filtration media contained in red blood cell filters manufactured by Asahi Medical Corporation—bearing the trade identifications RS-2000; RZ-400; Flex RC, etc.—compared to the residual leukocyte population and/or RBC recovery in red blood cells filtered by the same media mixed with conventional Adsol additive solution (manufactured by Baxter Healthcare Corporation). While the use of Adsol® additive solution prior to filtration results in acceptable reduced residual leukocyte populations and/or RBC recovery in pRBC's after filtration, Adsol® additive solution does not provide all the desired conditioning effects that Esol™ Solution provides. The use of Esol™ Solution is therefore still desired. Conversely, it has been observed that the residual leukocyte population and/or RBC recovery in packed red blood cells is not significantly different when the pRBC's are filtered mixed with the Esol-A component through the fibrous depth filtration media contained in red blood cell filters manufactured by Pall Corporation—bearing the trade identifications RCM-1, RC2D, BPF-4, etc.—compared to the residual leukocyte population in red blood cells filtered by the same media mixed with conventional Adsol® additive solution. It has also been observed that the residual leukocyte population and/or RBC recovery in packed red blood cells is not significantly different when the pRBC's are filtered mixed with the Esol-A component through the non-fibrous membrane filtration media contained in red blood cell filters manufactured by Terumo Corporation—bearing the trade identifications Imugard-III-RC—compared to the residual leukocyte population and/or RBC recovery in red blood cells filtered by the same media mixed with conventional Adsol® additive solution. To achieve the full benefits of dilution function 44, it is therefore desirable to first differentiate between filtration media having performance characteristics that are adversely affected by exposure to the Esol-A component 40 (which will be called Category A Filtration Media) from filtration media that is not adversely affected (which will be called Category B Filtration Media). This differentiation can be achieved by in vitro testing, as the following Example 1 demonstrates.

### EXAMPLE 1

### Identifying Category A and Category Filtration B Media

Units of pRBC's (270 ml) were obtained by conventional manual centrifugation techniques from whole blood. The pRBC's were mixed with conventional Esol™ Solution at a 2:1 volumetric ratio. The pRBC-Esol mixtures were passed through various filtration media at room temperature and with no hold time after Esol™ Solution addition. The following Table summarizes the data (PASS indicates that the residual leukocyte population and/or RBC recovery in red blood cells mixed with the Esol™ Solution met selected standards of numbers of residual leukocytes and/or percentage of RBC recovery that can be achieved when red blood cells are filtered mixed with Adsol® Solution—in this Example, the standards selected were (1) having a residual leukocyte level that was less than 1×106 per unit with 95% confidence, 95% of the time and (2) a RBC recovery that was not less than about 89%, assuming a minimum 270 ml RBC unit. FAIL indicates that the selected residual leukocyte population and/or RBC recovery standards were not met in red blood cells mixed with the Esol™ Solution):

TABLE 1
Filtration of pRBC's Mixed with Esol ™ Solution

| | | RBC | Residual | | |
|---|---|---|---|---|---|
| | | Recovery | Leukocyte | Filtration | |
| | | (%) | Population | Time | |
| | No. | Mean | (Mean) | (Mean | |
| Filter | Tested | Range | Range | Min) | Disposition |
| Asahi | 7 | 89 | 5.6 × 10⁴ | 11 | FAIL |
| RS-2000 | | 88 to 90 | 7.18E4 to | | Category A |
| | | | 1.26E6 | | |
| Asahi | 8 | 89 | 2.0 × 10⁶ | 18 | FAIL |
| Flex-RC | | 88 to 91 | 1.31E4 to | | Category A |
| | | | 1.10E7 | | |
| Miramed | 7 | 90 | 5.05 × 10⁸ | 11 | FAIL |
| Leuco- | | 89 to 91 | 1.29E8 to | | Category A |
| stop 4 | | | 1.19E9 | | |
| LDSS Mono | | | | | |
| Miramed | 5 | 90 | 7.71 × 10⁸ | 9 | FAIL |
| HE1 LDSS | | 89 to 91 | 3.2E8 to | | Category A |
| Mono | | | 1.64E9 | | |
| Hemasure | 5 | 93 | 1.73 × 10⁶ | 12 | FAIL |
| r/LS | | 92 to 93 | 8.28E4 to | | Category A |
| | | | 5.85E6 | | |
| Fresenius | 5 | 90 | 1.47 × 10⁷ | 9 | FAIL |
| BioR- | | 90 to 91 | 4.46E5 to | | Category A |
| 01MaxBS | | | 4.68E7 | | |
| Pall | 5 | 94 | 1.64 × 10⁵ | 17 | PASS |
| RCM1 | | 93 to 95 | 1.66E4 to | | Category B |
| | | | 4.89E5 | | |
| Pall | 10 | 88 | 6.94 × 10⁴ | 19 | PASS |
| RC2D | | 87 to 90 | 1.44E4 to | | Category B |
| | | | 1.5 E5 | | |
| Terumo | 22 | 89 | 4.84 × 10⁴ | 7 | PASS |
| Imugard | | 86 to 98 | 8.79E2 to | | Category B |
| III-RC | | | 5.32E5 | | |

It is believed that Category B Filtration Media may have inherently different mechanisms for leukocyte adhesion than Category A Filtration Media, which results in different filtration efficiencies under certain special conditions.

### 1. Using Category A Filtration Media

When a Category A Filtration Media is selected for use, the dilution function 44 desirably includes systems and related methods that mediate the exposure of the pRBC unit 16 to the Esol-A component 40 (or like component) prior to and/or during filtration. This mediation can be accomplished in various ways.

a. Lowering the Hypotonicity of Esol-A

A potential cause of the phenomenon discussed above for Catagory A Filtration Media is believed to be related to the hypotenicity of the conventional composition of the Esol-A component 40. Due to the absence of dextrose, the hypotonicity of the Esol-A component 40 is significantly greater than the hypotoncity of solutions containing dextrose, for example Adsol® Solution. Expressed in terms of osmolarity, the dextrose-free Esol-A component has an osmolarity of 178 mOsm/kg, whereas Adsol® Solution (containing 20.0 g/L of dextrose) has an osmolarity of 466 mOsm/kg). It is believed that the hypotenicity of the conventional composition of the Esol-A component due to the absence of dextrose may evoke physiologic changes in the morphology of the red blood cells and/or leukocytes, which ultimately affect the selective adsorption and/or flow dynamics of the Category A Filtration Media.

To make possible the synergistic coordination of the leukocyte reducing and conditioning functions 18 and 20, the composition of the Esol-A component 40 can be modified to present a lower hypotenicity. The lowered hypotenicity can be achieved in various ways. One such way is by adding a selected amount of D-glucose anhydrous or monohydrate (dextrose) to the other Esol-A ingredients.

### EXAMPLE 2

### Addition of Dextrose Leads to Improved Leukocyte Reduction During Filtration

pRBC's from a pooled source were mixed with various additive solutions at a 2:1 volumetric ratio. Filtration through an Asahi Flex RC™ filter (a Category A Filtration Medium) began five to six minutes after addition of solution. The following table shows the results:

TABLE 2
Filtration of pRBC's
Mixed with Esol-A Solution
Modified with Dextrose

| | | Modified Esol-A |
|---|---|---|
| | Esol-A Solution | Solution |
| Composition | (No Dextrose) | (With Dextrose) |
| Mannitol | 7.74 | 7.74 |
| g/L | | |
| Adenine | 0.215 | 0.215 |
| g/L | | |
| Dextrose | 0.0 | 20.0 |
| (anhydrous) | | |
| g/L | | |
| Sodium | 7.82 | 7.82 |
| Citrate | | |
| (dihydrate) | | |
| g/L | | |
| Sodium | .649 | .649 |
| Phosphate | | |
| (monohydrate) | | |
| g/L | | |
| Sodium | 2.42 | 2.42 |
| Phospate | | |
| Dibasic | | |
| (anhydrous) | | |
| Osmolarity | 178 | 293 |
| mOsm/kg | | |
| Initial | 1.86 × 10⁹ | 1.86 × 10⁹ |
| Leukocyte | | |
| Population | | |
| per 270 ml | | |
| Post- | 2.43 × 10⁵ | 4.05 × 10⁴ |
| Filtration | | |
| Leukocyte | | |
| Population | | |
| per 270 ml | | |

Table 2 demonstrates that the addition of dextrose to Esol-A Solution leads to improved leukocyte removal rates using a Category A Filtration Medium.

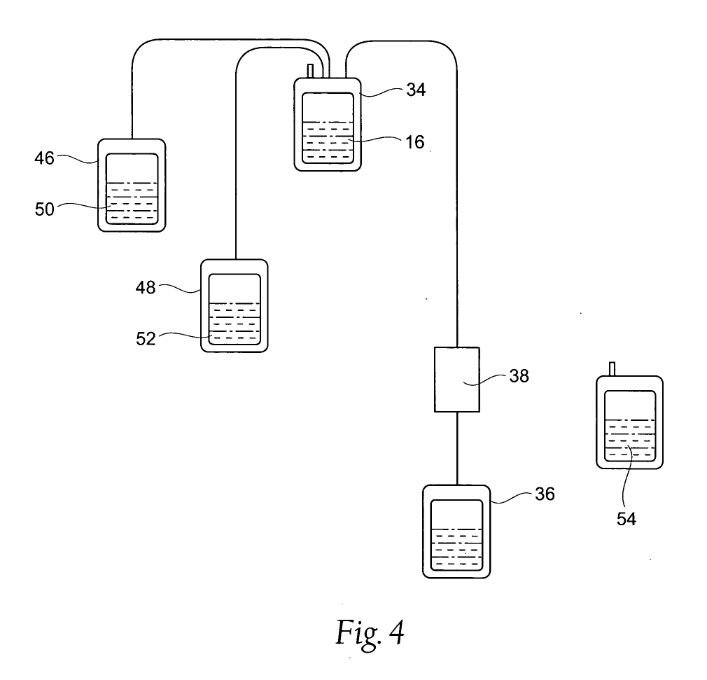

A representative composition for a hypotonicity-modified Esol-A component is: 94 ml of sodium citrate (26.6 mM); dibasic sodium phosphate (17.0 mM); monobasic sodium phosphate (4.7 mM); adenine (1.6 mM); mannitol (42.5 mM); and D-glucose monohydrate (20 to about 86 mM). Due to the heat sterilization considerations previously described, the modified Esol-A composition is desirably provided in two parts. In a representative embodiment shown in FIG. 4, two containers 46 and 48 can be coupled (either by an integral connection or by use of a sterile docking technique) to the collection container 34.

The container 46 holds a low pH D-glucose component 50—comprising, e.g., about 24 to 70 mL of (about 94/24 to 94/70) of D-glucose monohydrate (20 to about 86 mM). This component 50 can withstand heat sterilization.

The container 48 holds the remaining high pH components 52—comprising, e.g., about 50 to 70 mL of (about 94/50 to 94/70) of sodium citrate (26.6 mM); dibasic sodium phosphate (17.0 mM); monobasic sodium phosphate (4.7 mM); adenine (1.6 mM); and mannitol (42.5 mM).

As before described (and as shown in FIG. 4), the collection container 34 is itself connected to the transfer container 36 through the in-line leukocyte filter 38 (either by an integral connection or by use of a sterile docking technique). In this arrangement, the Esol-A components 50 and 52 are separately added to the pRBC unit 16 in the collection container 34 and mixed prior to filtration. It is the lowered viscosity mixture of Esol-A components 50 and 52 and red blood cells that is passed through the filter 38 into the transfer container 36.

In this arrangement, the composition of the Esol-B component is modified to result, after mixing with the modified Esol-A components 50 and 52, in the same overall final composition of the Esol conditioning solution 22. In the described embodiment, the modified Esol-B component 54 comprises 20 ml of D-glucose (0 to about 305 mM) in water. The 20 mL of the modified Esol-B component 54 can be used as the suspension agent for the pathogen inactivating compound 26 and quenching agent, as before described. This suspension is added to the pRBC's and high pH Esol-A components 50 and 52 residing in the container 36 during the subsequent mixing function 24, as has been previously described.

In this arrangement, all or part of the D-glucose desired in the ultimate conditioning solution 22 can be formulated in the modified low pH D-glucose component 50 in the container 46, as required to keep the hypotenicity of the mixed hypotonicity-modified Esol-A component at a desired state. It is believed that this lessened degree of hypotenicity for the Esol-A component (and/or the preservation effect of dextrose itself) will facilitate the selective removal of leukocytes by Category A Filtration Media. In the event that all of the D-glucose is formulated in the component 52 in the container 46, the water ingredient of the modified Esol-B component 54 (which would now be free of any D-glucose) could be partially or entirely replaced by a 0.9% saline or other salt solution. The substitution—entire or partial—of the saline or salt solution for water will “soften” the effects of adding the now more hypotonic Esol-B component resuspension of the pathogen inactivating compound 26 and quenching agent to the red blood cells.

Dextrose is known to rapidly cross cell membranes and therefore may not, on its face, be considered as contributing to “effective” osmolarity. Still, if the dextrose cannot equilibrate between the cells and the supernatent—and, as a result, the extracellular concentration remains greater than the intracellular concentration—then dextrose may provide some contribution to the “effective” osmolarity, even though it does not affect ionic strength.

To make possible the synergistic coordination of the leukocyte reducing function 18 and condition function 20, the composition of the S-Sol-A component 40 can be modified to increase its ionic strength, as well contribute to osmolarity, by the addition of sodium chloride to Esol-A.

### EXAMPLE 3

### Addition of Sodium Chloride Leads to Improved Leukocyte Reduction During Filtration

pRBC's from a pooled source were mixed with various additive solutions at a 2:1 volumetric ratio. Filtration through an Asahi Flex RC™ filter (a Category A Filtration Medium) began five to six minutes after addition of solution. The following table shows the results:

TABLE 3
Filtration of pRBC's
Mixed with Esol-A Solution

| | | Modified Esol- |
|---|---|---|
| Modified with | Esol-A Solution | A Solution |
| Sodium Chloride | (No Sodium | (With Sodium |
| Composition | Chloride) | Chloride) |
| Mannitol | 7.74 | 7.74 |
| g/L | | |
| Adenine | 0.215 | 0.215 |
| g/L | | |
| Sodium Chloride | 0.0 | 4.39 |
| g/L | | |
| Sodium Citrate | 7.82 | 7.82 |
| (dihydrate) g/L | | |
| Sodium Phosphate | .649 | .649 |
| (monohydrate) g/L | | |
| Sodium Phospate | 2.42 | 2.42 |
| Dibasic | | |
| (anhydrous) | | |
| Osmolarity | 178 | 315 |
| mOsm/kg | | |
| Initial Leukocyte | 1.86 × 10⁹ | 1.97 × 10⁹ |
| Population | | |
| per 270 ml | | |
| Post-Filtration | 2.43 × 10⁵ | 5.40 × 10⁴ |
| Leukocyte | | |
| Population | | |
| per 270 ml | | |

Table 3 demonstrates that the addition of sodium chloride to Esol-A Solution leads to improved leukocyte removal rates when using a Category A Filtration Medium. Centrifugation conditions also may have an impact upon the magnitude of leukocyte reduction. If pRBC's are separated from whole blood at greater centrifugal forces (a so-called “hard spin”), a majority of platelets sediment into the pRBC's. This is contrasted with the separation of pRBC's at lower centrifugation forces (a so-called “soft spin”), during which a majority of the platelets remain in the supernatant. It has been discovered that use of a “hard spin” to harvest pRBC's optimizes the synergistic coordination of the leukocyte reducing function 18 and condition function 20, particularly in conjunction with use of a dextrose-modified Esol-A Solution.

### EXAMPLE 4

### Centrifugation Conditions Affect Leukocyte Removal During Subsequent Filtration

Freshly collected whole blood was subject to centrifugation under both “hard spin” and “soft spin” conditions to yield pRBC's. The pRBC's were mixed with various additive solutions at a 2:1 volumetric ratio. Filtration through an Asahi Flex RC™ filter (a Category A Filtration Medium) began after addition of solution. The following table shows the results:

TABLE 4
Filtration of pRBC's
Mixed with Various Additive Solutions
After Hard Spin and Soft Spin Conditions

| | | | | Mean Residual |
|---|---|---|---|---|
| | Dextrose | Number | | Post |
| Additive | Concentration | of | | Filtration |
| Solution | in Additive | Units | Centrifugation | Leukocyte |
| Composition | Solution | Tested | Conditions | Population |
| Esol-A | 0 | 5 | Hard Spin | 3.26 × 10⁵ |
| Esol-A | 0 | 6 | Soft Spin | 1.52 × 10⁶ |
| Esol-A with | 35 mM | 4 | Hard Spin | 1.40 × 10⁵ |
| Dextrose | | | | |
| Esol-A with | 35 mM | 6 | Soft Spin | 4.10 × 10⁴ |
| Dextrose | | | | |
| Esol-A with | 71 mM | 5 | Hard Spin | 5.12 × 10⁴ |
| Dextrose | | | | |
| Esol-A with | 71 mM | 5 | Soft Spin | 6.39 × 10⁴ |
| Dextrose | | | | |
| CPD Plasma | Conventional | 5 | Hard Spin | 2.16 × 10⁵ |
| | Formulation | | | |

Table 4 demonstrates that leukodepletion of pRBC's can be enhanced by the presence of platelets, which is the result of a hard spin. Table 4 also underscores that the addition of dextrose improves leukocyte reduction performance of a Category A Filtration Medium.

As Table 4 also demonstrates, the pRBC unit may also be modified by retaining a greater volume of anticoagulated CPD plasma, resulting in improved removal of leukocytes using a Category A Filtration Medium. The presence of a greater volume of CPD or ACD plasma elevates the osmolarity (reducing hypotonicity) of the blood product, and also provides dextrose for metabolic function.

b. Use of a Less Hypotonic Solution During Filtration, Replaced by Esol-A After Filtration

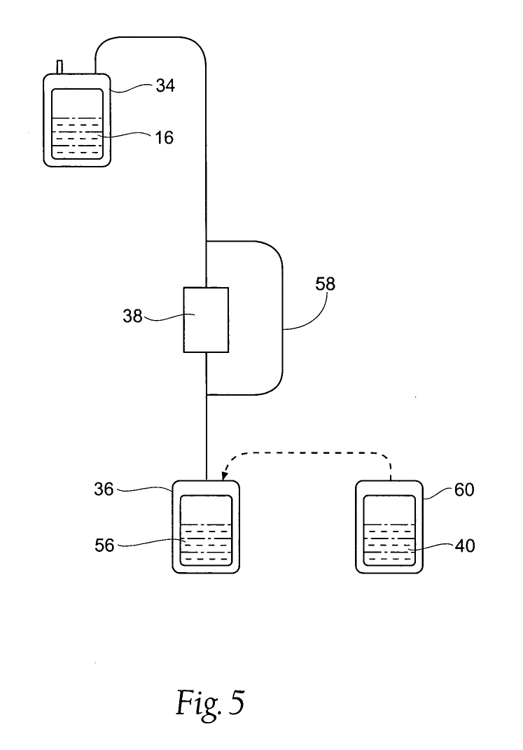

In another embodiment (see FIG. 5), a synergistic coordination of the leukocyte reducing and conditioning functions 18 and 20 is made possible by using a less hypotonic additive solution 56 to dilute the pRBC's during filtration using Category A Filtration Media. This additive solution 56 is selected, not necessarily to provide any conditioning for a subsequent pathogen inactivating function, but rather to provide an environment where optimal removal of leukocytes occurs during the leukocyte removal function 18 using the Category A Filtration Media. For example, the additive solution 56 can comprise conventional Adsol® Solution or SAG-M™ Solution.

In this arrangement, the conditioning function 20 includes the removal of the additive solution 56 following filtration and the subsequent addition of the Esol-A component 40, as currently formulated, to the filtered pRBC unit 16.

To carry out this process (as a representative embodiment of FIG. 5 shows), the collection container 34 for the pRBC unit 16 can be coupled to the transfer container 36 via the in-line filter 38, as previously described. In this representative embodiment, the transfer container 36 holds the non-conditioning additive solution 56. The additive solution 56 is transferred, e.g., by gravity flow through a one-way by-pass path 58, into the collection container 34. The additive solution 56 is mixed with the pRBC unit 16 in the collection container 34. After mixing, the pRBC unit 16 and additive solution 56 are conveyed through the filter 38 into the transfer container 36, e.g., by gravity flow. The transfer container 36 is separated from the filter 38, and the filtered pRBC and additive solution 56 are centrifugally separated within the transfer container 36. The additive solution 56 is expressed from the container 36 (e.g., into another container or—if the transfer container 36 is not disconnected from the container 34 before centrifugation—into the container 34, leading the filtered pRBC unit behind in the container 36. The Esol-A component 40, as presently formulated, can now be transferred into the transfer container 36 from an auxiliary container 60 where it is mixed with the pRBC unit 16. The auxiliary container 60 can be either integrally connected by tubing to the transfer container 36 or connected by a sterile docketing technique.

In this arrangement, the Esol-B component, as presently formulated, can be used as the suspension agent for the pathogen inactivating compound 26 and quenching agent, and added to the pRBC's and high pH Esol-A component 40 residing in the container 36 during the subsequent mixing function 24, as has been previously described.

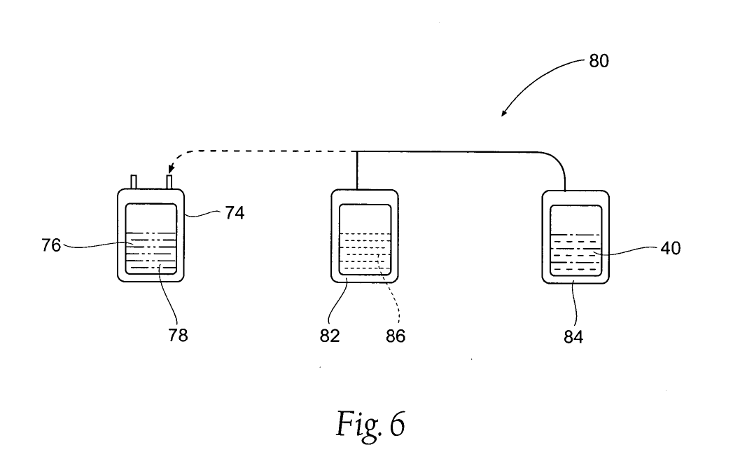

In an alternative embodiment shown in FIG. 6, a collection container 74 may include a pre-processed pRBC unit 76 that has already undergone leukocyte reduction and to which an additive solution 78 (like Adsol® Solution) has already been added. The pre-processed unit 76 may be provided, e.g., as a result of processing by the automated Alyx™ Blood Separation System, or by the automated Trima™ Blood Separation System sold by Cobe Laboratories (a division of Gambro), or by the MSC Plus™ Blood Separation System sold by Haemonetics Corporation. It may be desirable to subject the pre-processed pRBC unit 76 to the conditioning function 24, to convert the pre-processed, leukocyte-reduced unit 76 into a pRBC unit 16 conditioned for a subsequent pathogen inactivation function 30.

In this embodiment, a conditioning conversion assembly 80 can be provided comprising an integrally connected empty container 82 and a container 84 holding the Esol-A component 40. The conditioning conversion assembly 80 is integrally attached or coupled to the collection container 74 by a sterile docking technique, after the collection container 74 has undergone centrifugation to separate the additive solution 78 from the pre-processed pRBC's. The centrifugally separated additive solution 78 is conveyed into the empty container 82, and the Esol-A component 40 is transferred into the collection container 74 to resuspend the pRBC's.

In an alternative arrangement, it may be desirable to add additional solution to the pre-processed pRBC unit 76 to decrease the viscosity of the pRBC's before subjecting the unit 76 to centrifugal separation. In this arrangement, the empty container 82 could hold a supplemental volume of an additive solution 86 (shown in phantom lines in FIG. 6) B e.g. Adsol® Solution or the modified low pH D-glucose component 50, described above. This solution 86 is added to the pRBC unit 76, and the unit 76 is then subject to centrifugal separation. Following centrifugal separation, the additive solution 78 and supplemental solution 86 are conveyed into the container 82 (which is now empty), and the Esol-A component 40 is conveyed from the container 84 into the collection container 74.

The Esol-B component, as presently formulated, can subsequently be used as the suspension agent for the pathogen inactivating compound 26 and quenching agent, and added to the pRBC's and high pH Esol-A component 40 residing in the container 74 during the subsequent mixing function 24, as has been previously described.

c. Elevating the Extracellular pH

A potential cause of the phenomenon discussed above for Catagory A Filtration Media is believed to be related to the extracellular pH of pRBC's in the presence of the conventional composition of the Esol-A component 40. pRBC's that are suspended in the Esol-A component 40 exhibit a lower extracellular pH (by approximately 0.1 to 0.2 pH units) than do pRBC's suspended in Adsol® Solution. It is believed that the lower pH may evoke physiologic changes in the morphology of the red blood cells and/or leukocytes, which ultimately affect the selective adsorption and/or flow dynamics of the Category A Filtration Media.

To make possible the synergistic coordination of the leukocyte reducing and conditioning functions 18 and 20, the composition of the Esol-A component 40 can be modified to present a high pH. The pH may be elevated, e.g., by revising the content of phosphate frpm 4.7 mM sodium phosphate monobasic and 17.0 mM sodium phosphate dibasic to approximately 21.7 mM sodium phosphate dibasic. The ph also may be elevated, e.g., by chilling the pRBC's prior to filtration. Chilling red blood cells raises the pH of the blood product.

d. Use of a Pump to Reduce Time of Exposure to Hypotonic Conditions During Filtration

The leukocyte removing function 18 can rely upon gravity flow to convey the pRBC unit 16 through the filter 38, as previously described. Even when the viscosity of the pRBC unit 16 is reduced by the addition of the Esol-A component 40 or other additive solution before filtration, the gravity flow filtration time can be as much as 30 minutes for pRBC's (240 mL to 360 mL) at ambient temperatures and as much as 360 minutes for pRBC's (240 mL to 360 mL) that have been refrigerated. The gravity flow filtration time also reflects the time that pRBC's are exposed to the Esol-A component 40.

Regardless of the exact mechanism that is causing the observed degradation of filtration efficiencies for Category A Filtration Media, it is believed that a correlation exists between the time of exposure of the pRBC's to the component 40 prior to filtration and changes in filtration efficiencies that may result as a result of that exposure during filtration. It is therefore believed desirable to shorten the time of filtration, to thereby shorten the time of exposure. Shortening the filtration time is advantageous not only with respect to facilitating the use of Category A Filtration Media, but it also shortens the overall processing time, which is beneficial regardless of the type of filtration media that is selected for use.

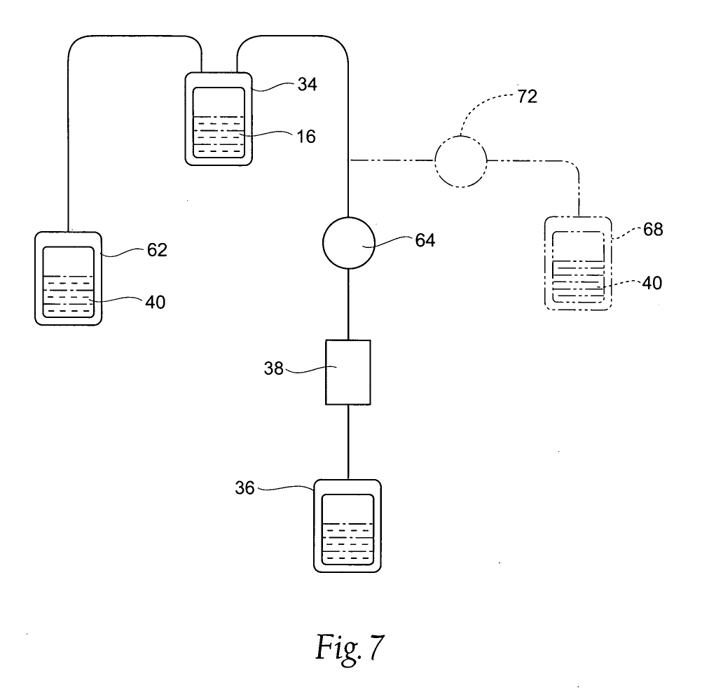

In a representative embodiment shown in FIG. 7), a synergistic coordination of the leukocyte reducing and conditioning functions 18 and 20 is made possible by using a pump 64 during the leukocyte reducing function 20. The pump 64 conveys the pRBC unit 16 from the collection container 34 through the filter 38 and into the transfer container 36 at flow rates in excess of gravity flow rates. The pump 64 may comprise, e.g., a convention peristaltic pump or a diaphragm pump or a syringe-type pump. In this embodiment, an auxiliary container 62 can be coupled (either by an integral connection or by use of a sterile docking technique) to the collection container 34. The container 62 holds the Esol-A component 40, as presently formulated. All or part of the Esol-A component 40 is conveyed into the collection container 34 (e.g., by gravity flow) for mixing with the pRBC unit 16 just prior to the commencement of filtration. The pump 64 is actuated to pump the pRBC unit 16 and mixed component 40 through the filter 38 and into the transfer container 36 at a commanded flow rate. If only a portion of the Esol-A component 40 is placed into the collection container 34 prior to filtration, the remainder of the Esol-A component 40 may be conveyed through the filter 38 after filtration of the pRBC unit 16 is completed, to perform a filter-rinsing step in the process of being mixed with the filtered pRBC unit 16.

Typically, commanded blood flow rates of upwards to 250 ml/min can be achieved using pumps without damage or hemolysis to red blood cells. Given a typical manually collected pRBC unit volume of 240 mL to 360 mL, the filtration time, and thus the overall time of exposure of the pRBC unit 16 to the conditions of the Esol-A component 40 during filtration, can be significantly reduced to less than two minutes.

Use of the pump 64 also promotes full exposure of the pRBC's to the entire surface area of the filtration media. More optimal leukocyte removal efficiencies can therefore be achieved, while also achieving the viscosity reducing benefits that the Esol-A component 40 provides, as well as also achieving the overall pathogen inactivation conditioning benefits that the combined Esol Solution provides. These benefits accrue from use of the pump 64 regardless of the type of filtration media that is selected for use.

Improved leukocyte-reduction may also result in the pump-driven arrangements just described due to the effect of pump-induced shear forces on the blood, which can stimulate platelet and/or leukocyte adhesion to the filtration media.

e. Metered Introduction of Hypotonic Esol-A Solution During Filtration

As previously described, about 94 ml of the Esol-A component 40 or other additive solution is added to the pRBC unit 16 before filtration to lower the viscosity of the pRBC unit 16 during filtration. The preceding embodiments mix the complete volume of the component 40 or additive solution to the pRBC unit 16 at one time before filtration. It is believed that the onset of reduced filtration efficiencies as a result of the exposure to the component 40 can also be mediated by metering the exposure of the pRBC's to the component 40 prior to and/or during filtration.

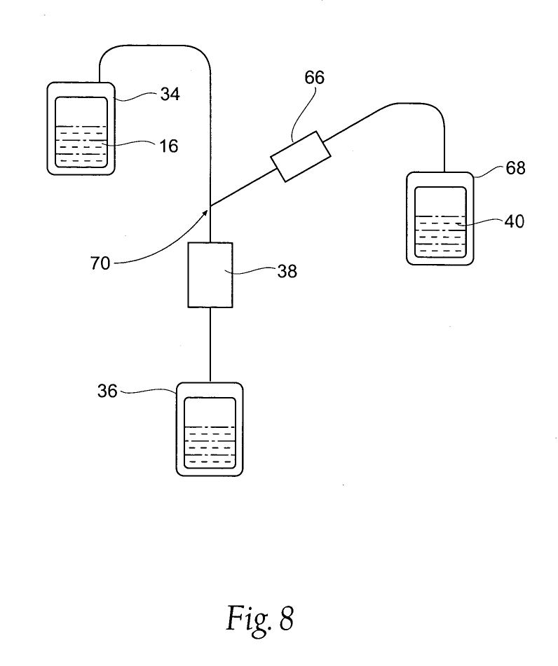

As shown in a representative embodiment in FIG. 8, the metering can be accomplished in a gravity flow system by conveying the Esol-A component 40 from a container 68 in a metered flow into the pRBC's prior to passage through the filter 38. The metered flow can be controlled, e.g., by an in-line manual or proportional flow restrictor device 66 located between the container 68 and the junction 70 at which the Esol-A component 40 enters the flow of pRBC's through the filter 38. The device 66 is desirably adjustable to select a desired flow rate ratio between pRBC's and Esol-A component 40 entering the filter 38.

As shown in phantom lines in FIG. 7, the metering can also be accomplished in a pump-assisted system. In this arrangement (shown in phantom lines), the Esol-A component 40 is conveyed from a container 68 at a controlled flow rate through a pump 72 into the pRBC's prior to passage through the filter 38. The flow rate of the pump 72 is desirably controlled relative to the flow rate of the pRBC pump 64 to provide the desired flow rate ratio between pRBC's and Esol-A component 40 entering the filter.

The metering of the Esol-A component 40 significantly reduces the time that pRBC's are exposed to the conditioning component 40 prior to filtration. Removal of leukocytes can therefore take place prior to the degradation of filtration efficiencies that may result due to that exposure.

Other advantages are obtained by the metering of the conditioning solution 22 into the pRBC's during the leukocyte reducing function 18, regardless of the type of filtration media that is selected for use. By controlling the ratio of pRBC's and conditioning solution, the conditioning solution is always introduced at a constant desired ratio. Therefore, regardless of the volume of red blood cells collected, the final red blood cell/conditioning solution hematocrit can be constant. The metered supply of red blood cells and conditioning solution through the filter 38 eliminates the need to first drain the conditioning solution into the red blood cell collection container 34, which lessens the overall procedure time. The metered supply of red blood cells and conditioning solution through the filter 38 also eliminates the need to manually agitate a red blood cell/conditioning solution mixture prior to leukofiltration. Due to density differences, when concentrated red blood cells are added to a conditioning solution, or vice versa, the conditioning solution floats to the top. Poorly mixed, high hematocrit, high viscosity red blood cells lead to reduced flow rates during leukofiltration. Poorly mixed, high hematocrit, high viscosity red blood cell conditions can also lead to hemolysis. By metering passage of red blood cells and conditioning solution through the filter 38, mixing occurs automatically without operator involvement.

### 2. Using Category B Filtration Media

Category B Filtration Media can be identified using the characterization process previously described. The coordination of the conditioning function 20 and the leukocyte removing function 18 above described can thereby proceed using the Category B Filtration Media, without regard to mediating the exposure of the pRBC unit 16 to the Esol-A component 40 or like component prior to and/or during filtration, as above described. Category B Filtration Media, e.g., the Terumo RC IIIC, can be used in blood collection systems of the type shown in FIGS. 2 and 3, to carry out the leukocyte reducing function shown in FIG. 1, with or without the dilution function 44.

Furthermore, the additional benefits of pump-assisted flow through the filter 68 and metering of conditioning solution into the pRBC's during leukocyte filtration remain desirable in their own right, even when using a Category B Filtration Media. Accordingly, Category B Filtration Media can be used in blood collection systems of the type shown in FIGS. 7 and 8.

It should now be apparent that the system and associated method 10 shown in FIG. 1 lends itself to practice in either a manual way (e.g., by gravity flow and manual manipulation of the containers), or an automated way (e.g., using pump-assisted flow and in-line flow control devices coupled to the containers), or in a hybrid way incorporating both manual and automated control techniques.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

1. In a method of processing a red blood cell product for pathogen inactivation, in which a synthetic conditioning solution is added to the red blood cell product to condition the red blood cell product for pathogen inactivation in the presence of a selected pathogen inactivating compound, the improvement comprising the steps of
selecting a filtration medium that removes leukocytes from red blood cells,
conveying the red blood cells product, prior to undergoing pathogen inactivation, through the selected filtration medium while diluted with at least one component of the synthetic conditioning solution and while free of the pathogen inactivating compound, and
the selection step being based, at least in part, upon an assessment that the removal of leukocytes from red blood cells by the filtration medium will not be significantly altered due to the presence of the at least one component.

2. A method according to claim 1, wherein the at least one component comprises Esol-A, and wherein the selected filtration medium comprises a red blood cell filter corresponding to a filter sold by Pall Corporation bearing the trade identification RCM-1 or counterparts or improvements thereof.

3. A method according to claim 1, wherein the at least one component comprises Esol-A, and wherein the selected filtration medium comprises a red blood cell filter corresponding to a filter sold by Terumo Corporation bearing the trade identification RC-IIIC or counterparts or improvements thereof.

4. A method according to claim 1, wherein, during the conveying step, a pump conveys the red blood cell product through the selected filtration medium.

5. In a method of processing a red blood cell product for pathogen inactivation, in which a synthetic conditioning solution is added to the red blood cell product to condition the red blood cell product for pathogen inactivation in the presence of a selected pathogen inactivating compound, the improvement comprising the steps of
selecting a filtration medium that removes leukocytes from red blood cells,
conveying the red blood cell product, prior to undergoing pathogen inactivation, through the selected filtration medium while diluted with at least one component of the synthetic conditioning solution and while free of the pathogen inactivating compound, and
the at least one component and/or the red blood cell product being manipulated prior to and/or during the conveying step to sustain removal of leukocytes from red blood cells by the filtration medium in the presence of the at least one component.

6. In a method of processing a red blood cell product for pathogen inactivation, in which a synthetic conditioning solution is added to the red blood cell product to condition the red blood cell product for pathogen inactivation in the presence of a selected pathogen inactivating compound, the improvement comprising the steps of
selecting a filtration medium that removes leukocytes from red blood cells,
conveying the red blood cell product, prior to undergoing pathogen inactivation, through the filtration medium while diluted with at least one component of the synthetic conditioning solution and while free of the pathogen inactivating compound,
the selecting step including an assessment that the removal of leukocytes from red blood cells by the filtration medium may be significantly altered due to the presence of the at least one component, and, based upon this assessment, the at least one component and/or the red blood cell product are manipulated prior to and/or during the conveying step to improve removal of leukocytes from red blood cells by the filtration medium.

7. A method according to claim 5 or 6, wherein the at least one component is manipulated to increase osmolarity of the at least one component prior to and/or during the conveying step.

8. A method according to claim 5 or 6, wherein the at least one component is manipulated by the addition of dextrose prior to and/or during the conveying step.

9. A method according to claim 5 or 6, wherein the red blood cell product is manipulated by increased retention of anticoagulated plasma prior to and/or during the conveying step.

10. A method according to claim 5 or 6, wherein the at least one component is manipulated to increase the pH of the at least one component prior to and/or during the conveying step.

11. A method according to claim 5 or 6, wherein the at least one component is manipulated to change a sodium chloride of the at least one component prior to and/or during the conveying step.

12. A method according to claim 5 or 6, wherein the at least one component is manipulated to change a phosphate content of the at least one component prior to and/or during the conveying step.

13. A method according to claim 5 or 6, wherein the red blood cell product is manipulated by chilling the red blood cell product prior to and/or during the conveying step.

14. A method according to claim 5 or 6, wherein, during the conveying step, a pump conveys the red blood cell product through the selected filtration medium.

15. A method according to claim 5 or 6, wherein the at least one component comprises Esol-A, and wherein the selected filtration medium comprises a red blood cell filter sold by Asahi Medical Corporation bearing the trade identification RS-2000, or RZ-400, or Flex RC, or improvements thereof.

16. A packed red blood cell unit comprising
a red blood cell concentrate volume, and
a synthetic conditioning solution that includes components that, when mixed, condition the red blood cell concentrate volume for pathogen inactivation in the presence of a selected pathogen inactivating compound,
the packed red blood cell unit being in a leukocyte-reduced condition as a result of filtration of the red blood cell concentrate volume while diluted with at least one component of the synthetic conditioning solution and while free of the pathogen inactivating compound.

17. A packed red blood cell unit comprising
a red blood cell concentrate volume, and
a synthetic conditioning solution that includes components that, when mixed, condition the red blood cell concentrate volume for pathogen inactivation in the presence of a selected pathogen inactivating compound,
the packed red blood cell unit being in a leukocyte-reduced condition as a result of filtration of the red blood cell concentrate volume while diluted with less than all the components of the synthetic conditioning solution and while free of the pathogen inactivating compound.

18. A packed red blood cell unit according to claim 16 or 17
further including a pathogen inactivating compound added to the red blood cell concentrate volume while suspended in a component of the synthetic conditioning solution.

19. A packed red blood cell unit according to claim 16 or 17
wherein the synthetic conditioning solution includes a dextrose component and a hypotonic component, and
wherein the red blood cell concentrate volume is filtered while diluted with the hypotonic component and not the dextrose component.

20. A packed red blood cell unit according to claim 19
wherein the hypotonic component is substantially free of dextrose.

21. A packed red blood cell unit according to claim 20
wherein the hypotonic component includes sodium citrate, sodium phosphate, adenine, and mannitol.

22. A packed red blood cell unit according to claim 19
further including a pathogen inactivating compound added to the red blood cell concentrate volume while suspended in the dextrose component and not the hypotonic component.

23. A packed red blood cell unit according to claim 16 or 17
wherein the red blood cell concentrate volume is filtered at a flow rate greater than gravity flow.

24. A packed red blood cell unit according to claim 16 or 17
wherein the red blood cell concentrate volume is filtered by gravity flow.

25. A packed red blood cell unit according to claim 16 or 17
wherein the components include sodium citrate, sodium phosphate, adenine, and mannitol.

26. A packed red blood cell unit according to claim 25
wherein the components further include dextrose.

27. A method of preparing a packed red blood cell unit comprising the steps of
collecting a red blood cell concentrate volume, and
providing a synthetic conditioning solution that includes components that, when mixed, condition the red blood cell concentrate volume for pathogen inactivation in the presence of a selected pathogen inactivating compound, and
filtering the red blood cell concentrate volume to remove leukocytes while the red blood cell concentrate volume is diluted with at least one component of the synthetic conditioning solution and while free of the pathogen inactivating compound, to thereby provide, after filtering, a packed red blood cell unit.

28. A method of preparing a packed red blood cell unit comprising the steps of
collecting a red blood cell concentrate volume, and
providing a synthetic conditioning solution that includes components that, when mixed, condition the red blood cell concentrate volume for pathogen inactivation in the presence of a selected pathogen inactivating compound, and
filtering the red blood cell concentrate volume to remove leukocytes while the red blood cell concentrate volume is diluted with less than all the components of the synthetic conditioning solution and while free of the pathogen inactivating compound, to thereby provide, after filtering, a packed red blood cell unit

29. A method according to claim 27 or 28
further including adding a pathogen inactivating compound to the red blood cell concentrate volume while suspended in a component of the synthetic conditioning solution.

30. A method according to claim 27 or 28
wherein the synthetic conditioning solution includes a dextrose component and a hypotonic component, and
wherein the red blood cell concentrate volume is filtered while diluted with the hypotonic component and not the dextrose component.

31. A method according to claim 30
wherein the hypotonic component is substantially free of dextrose.

32. A method according to claim 31
wherein the hypotonic component includes sodium citrate, sodium phosphate, adenine, and mannitol.

33. A method according to claim 30
further including adding a pathogen inactivating compound to the red blood cell concentrate volume while suspended in the dextrose component and not the hypotonic component.

34. A method according to claim 27 or 28
wherein the red blood cell concentrate volume is filtered at a flow rate greater than gravity flow.

35. A method according to claim 27 or 28
wherein the red blood cell concentrate volume is filtered by gravity flow.

36. A method according to claim 27 or 28
wherein the components include sodium citrate, sodium phosphate, adenine, and mannitol.

37. A method according to claim 36
wherein the components further include dextrose.

38. In a method of processing a red blood cell product for pathogen inactivation, in which a synthetic conditioning solution is added to the red blood cell product to condition the red blood cell product for pathogen inactivation in the presence of a selected pathogen inactivating compound, the improvement comprising the step using a pump to convey the red blood cell product through a filtration medium to filter leukocytes from the red blood cell product while diluted with at least one component of the synthetic conditioning solution and while free of the pathogen inactivating compound.

39. In a method of processing a red blood cell product for pathogen inactivation, in which a synthetic conditioning solution is added to the red blood cell product to condition the red blood cell product for pathogen inactivation in the presence of a selected pathogen inactivating compound, the improvement comprising the steps of subjecting whole blood to a centrifugation condition to obtain a platelet-rich red blood cell product, and conveying the platelet-rich red blood cell product through a filtration medium to filter leukocytes from the platelet-rich red blood cell product while diluted with at least one component of the synthetic conditioning solution and while free of the pathogen inactivating compound.

40. A method according to claim 38 or 39, wherein the at least one component includes dextrose.

41. A method according to claim 38 or 39 wherein the at least one component includes anticoagulated plasma.

42. A method according to claim 38 or 39, wherein the at least one component includes sodium chloride.

43. A method according to claim 38 or 39 wherein the at least one component includes a phosphate content.

44. A method according to claim 38 or 39, wherein the platelet-rich red blood cell product is manipulated by chilling the platelet-rich red blood cell product prior to and/or during the conveying step.

45. A method according to claim 39, wherein, during the conveying step, a pump conveys the platelet-rich red blood cell product through the filtration medium.
